(19) United States
(12) Patent Application Publication
(10) Pub. No.: US 2021/0196630 A1
(43) Pub. Date: Jul. 1, 2021

(54) MULTICOMPARTMENT SYSTEM OF NANOCAPSULE-IN-NANOCAPSULE TYPE, FOR ENCAPSULATION OF A LIPOPHILIC AND HYDROPHILIC COMPOUND, AND THE RELATED PRODUCTION METHOD

(71) Applicant: Krzysztof Smela, Rzeszów (PL)

(72) Inventors: Krzysztof SMELA, Rzeszów (PL); Szczepan ZAPOTOCZNY, Kraków (PL); Joanna SZAFRANIEC, Kraków (PL)

(21) Appl. No.: 17/268,652

(22) PCT Filed: Aug. 19, 2019
(86) PCT No.: PCT/PL2019/000069

§ 371 Date: Feb. 16, 2021

(30) Foreign Application Priority Data
Aug. 17, 2018 (PL) P.426702
Jul. 16, 2019 (PL) P.430624

(51) Int. Cl. A61K 9/113; A61K 31/728; A61K 38/28; A61K 9/51

(52) U.S. Cl. CPC A61K 9/113; A61K 31/728; B82Y 5/00; A61K 9/51; A61K 38/28

(57) ABSTRACT

A multicompartment system of nanocapsule-in-nanocapsule type based on hyaluronic acid derivative is designed for encapsulation of peptides and/or hydrophobic active compounds, either simultaneously or separately, where surfactants, emulsifiers and/or stabilizers are not required for the system stability. The system functions as a carrier which enables protection of sensitive hydrophilic substances against aggressive external environment, and the resulting degradation and deactivation, and makes it possible to concurrently administer active substances of varied hydrophilicity. A method is provided of producing a multicompartment nanocapsule-in-nanocapsule system in the form of water-in-oil-in-water double emulsion.

### AREA OF TECHNOLOGY

The object of the invention is a multicompartment system of nanocapsule-in-nanocapsule type, for encapsulation of a lipophilic and hydrophilic compound, and the related production method based on water-in-oil-in-water (W/O/W) double emulsion, stabilized with a hydrophobized derivative of hyaluronic acid, presenting no need to use additional emulsifiers, the said system being a carrier, which also solves a problem related to the need to ensure protection of sensitive hydrophilic substances including proteins, against aggressive external environments, and enables concurrent administration of active substances of varied hydrophilicity.

### STATE OF TECHNOLOGY

A need to simultaneously apply hydrophobic and hydrophilic compounds is frequently linked with synergistic action of combinations of active substances (Chou T C (2006) Theoretical basis, experimental design, and computerized simulation of synergism and antagonism in drug combination studies. Pharmacol Rev 58: 621-681; Zimmermann G R, Lehar J, Keith C T (2007) Multi-target therapeutics: when the whole is greater than the sum of the parts. Drug discovery today 12: 31-42.) or with a possibility of concurrent and colocalized delivery of therapeuticals and substances supporting the diagnostic process (theranostics) (Liu G, Deng J, Liu F, Wang Z, Peerc D, Zhao Y, Hierarchical theranostic nanomedicine: MRI contrast agents as a physical vehicle anchor for high drug loading and triggered on-demand delivery, J. Mater. Chem. B, 2018, 6, 1995-2003). This is, in particular, related to administration of medication, vitamins, hormones and contrast agents in magnetic resonance imaging, etc. In the case of drug administration is it especially important in treatment of complex diseases, such as cancer (Blanco E et al. Colocalized delivery of rapamycin and paclitaxel to tumors enhances synergistic targeting of the PI3K/Akt/mTOR pathway. Mol Ther. 2014 July; 22(7):1310-1319.), or in combatting drug resistance in microbes and fungi (Levy S B, Marshall B (2004) Antibacterial resistance worldwide: causes, challenges and responses. Nature medicine 10: 2 S122-129; Fitzgerald J B, Schoeberl B, Nielsen U B, Sorger P K (2006) Systems biology and combination therapy in the quest for clinical efficacy. Nature chemical biology 2: 458-466).

The applied active substances of varied hydrophilicity usually differ in terms of pharmacokinetics, which adversely impacts synergistic effects in the body, even if a mixture of such substances is administered concurrently. The problem may be solved by administration of such substances in one submicrometer-size carrier which will deliver both (or many) substances concurrently to one location (colocalization). Such carriers may be based on systems of water-in-oil-in-water double emulsions, and structurally they can be described as a capsule with water core embedded in a capsule with oil core, like in the current invention.

In the case of hydrophilic compounds, the protective effect achieved by isolating the substance from the external environment is also of significance because the latter may destroy the substance (e.g. gastric juice with low pH, lymphocytes responsible for the body's immune response). This particularly relates to oral delivery of proteins and peptides (Abdul Muheem, Faiyaz Shakeel, Mohammad Asadullah, Jahangir, Mohammed Anwar, Neha Mallick, Gaurav Kumar Jain, Musarrat HusainWarsi, Farhan Jalees Ahmad, A review on the strategies for oral delivery of proteins and peptides and their clinical perspectives, Saudi Pharmaceutical Journal 2016, 24, 413-428).

Bioavailability of biologically active substances is determined by the rate and the range of their absorption [US Food and Drug Administration. Code of federal regulation. Title 21, volume 5, chapter 1, subchapter D, part 320. Bioavailability and bioequivalence reagents]. Low biological availability of a drug means that the medication will fail to achieve minimal effective concentration in blood, and consequently it will be difficult to produce the desirable therapeutic effects. The inability of the substance to reach and/or accumulate in a required location leads to a necessity to increase the dose, and that consequently may produce unwanted side effects and lead to higher costs of the therapy. Due to the above factors, only one in nine newly synthesized substances are approved by regulatory bodies [Blanco E. et al., Nat. Biotechnol. 2015, 33, 941-951].

The methods applied to improve bioavailability include production of prodrugs, solid dispersions with polymer carriers, micronization of substance particles or addition of surfactants [Baghel, S. et al., Int. J. Pharm. 2016, 105, 2527-2544]. Over the recent years a lot of focus has also been placed on micro- and nano-carriers, in particular in relation to poorly water-soluble substances [Chen H., et al., Drug Discov. Today. 2010, 7-8 354-360]. Nanonization leads to increased solubility and improved pharmacokinetics of the therapeutic substance; it also contributes to reducing adverse side effects of the substance uptake. The comprehensively investigated carriers include nanoemulsions, micelles, liposomes, self-emulsifying systems, solid lipid nanoparticles and polymer-drug conjugates [Jain S. et al., Drug Dev. Ind. Pharm. 2015, 41, 875-887].

Research has shown that the use of nanocarriers does not only result in improved pharmacokinetic parameters and better protection of sensitive substances against degradation, but also extends the duration of circulation and ensures targeted delivery of the active substance. Resulting from advancements in research focusing on drug delivery systems, the options today available in the market include nanoparticle formulations used in treatment of fungal infections, hepatitis A, and multiple sclerosis [Zhang L., et al. Clin. Pharmacol. Ther. 2008, 83, 761-769]. The first drug based on a nanoformulation was the liposomal form of doxorubicin (Doxil), designed for treatment of Kaposi's sarcoma, and approved by the U.S. Food and Drug Administration in 1995 [Barenholz Y. J. Control. Release 2012, 160, 117-134]. Ten years later approval was obtained for another formulation, i.e. nanoparticle albumin-bound paclitaxel (Abraxane). In this case, by eliminating the use of Cremophor EL it was possible to reduce harmful side effects associated with the conventional paclitaxel formulation.

Carrier systems for hydrophobic or lipophilic substances are mainly intended to improve pharmaceutical and biological availability of these substances. In the case of hydrophilic compounds, the protective effect achieved by isolating the substance from the external environment is also of significance because the latter may destroy the substance (e.g. gastric juice with low pH, lymphocytes responsible for the body's immune response). This particularly relates to oral delivery of proteins and peptides [Muheem A. et al., Saudi Pharm. J. 2016, 24, 413-428].

Insulin is the main protein hormone synthetized by β cells of pancreatic islets of Langerhans, necessary in treatment of type 1 diabetes. Given its prevalence, diabetes is globally one of the most widespread noncommunicable diseases [Shah R. B. et al., Int. J. Pharm. Investig. 2016, 6, 1-9]. Insulin is most commonly injected subcutaneously, which in many cases is associated with poor glycemic control, a sense of discomfort and deterioration of lifestyle [Owens D. R. Nat. Rev. Drug Discov. 2002, 1, 529-540]. Oral insulin delivery would be the most comfortable and preferential method of the hormone administration. Furthermore, oral delivery of the hormone would facilitate its absorption into hepatic portal circulation, imitating the physiological route for supplying insulin to the liver, and decreasing the systemic hyperinsulinemia linked with subcutaneous injection which delivers insulin to peripheral circulation, and possibly minimizing a risk of hypoglycemia and improving metabolic control [Heinemann L. and Jacques Y. J. Diabetes Sci. Technol. 2009, 3, 568-584].

The main barriers to intestinal absorption of insulin include the low permeability of proteins in the intestinal wall, as well as high susceptibility to denaturation in the acidic gastric environment and to enzymatic degradation in the intestine. A number of strategies for improving absorption of insulin in the digestive tract, so far published in the literature, include encapsulation of insulin in nanospheres or nanoparticles, microparticles and liposomes. These carriers protect the peptide against the proteolytic/denaturation processes in the upper part of the digestive tract and enable increased transmucosal protein capture in various parts of the small intestine. However, the use of the carriers is limited due to the poor effectiveness of encapsulation, and lack of control regarding release kinetics of active substance [Song L. et al., Int. J. Nanomedicine 2014, 9, 2127-2136; Sajeesh S. and Sharma C. P. J. Biomed. Mater. Res. B Appl. Biomater. 2006, 76, 298-305; Sarmento B. et al., Biomacromolecules. 2007, 8, 3054-3060; Niu M et al., Eur. J. Pharm. Biopharm. 2012, 81, 265-272].

Polish patent number PL229276 B discloses stable oil-in-water (O/W) systems with a core-shell structure, stabilized with modified polysaccharides, and able to effectively encapsulate hydrophobic compounds.

International patent no. WO 2016/179251 presents stable emulsions able to encapsulate volatile chemical compounds, e.g. derivatives of cyclopropane. Water-in-oil-in-water double emulsion contains an emulsifier and a surfactant ensuring its stability.

Stable double emulsions are described in the American patent US 2010/0233221. They contain a minimum of two emulsifiers with varied molar mass which ensure stabilization of water-in-oil emulsion and double emulsion.

International patent WO 2018/077977 presents double emulsions containing cross-linked fatty acids, as an inner layer, intended to encapsulate hydrophilic compounds used in cosmetics. The emulsions are stable for a minimum of three months.

International patent no. WO 2017/199008 describes double emulsions containing emulsifiers and inner aqueous phase comprising polymers subject to cross-linking at elevated temperatures, as a result of which hydrogel-in-oil-in-water systems are obtained. The obtained systems are able to carry active substances (drugs and cells) incorporated in hydrocolloid particles.

Stable double emulsions are described in American patent no. US 2010/0233221. They contain a minimum of two emulsifiers with varied molar mass which ensure stabilization of water-in-oil emulsion and double emulsion.

American description US20170360894 discloses production of an oral form of insulin involving production of a bolus containing an agent neutralizing acidic gastric environment as well as a self-emulsifying protein containing system.

Patent description U.S. Pat. No. 6,191,105 presents water-in-oil (W/O) emulsion systems containing insulin. However, oral delivery of the formulation may lead to a phase transition within the emulsion system, which may lead to untimely release of the peptide and its degradation in the digestive tract.

As revealed in American patent no. U.S. Pat. No. 6,277,413, in a formulation of polymer- and lipid-containing microspheres, insulin is encapsulated in the internal aqueous phase, however effectiveness of such encapsulation is very low.

Production of a polysaccharide insulin carrier was described in U.S. Ser. No. 09/828,445. Chitosan nanoparticles are produced by cross-linking of chitosan previously subjected to amidation with a fatty acid, a modified fatty acid and/or an amino acid. Insulin, on the other hand, is adsorbed onto the carrier.

Chitosan is also used in production of W/O/W systems for protein encapsulation and oral administration. Nanocarriers disclosed in the description CN106139162 additionally contain polygalacturonic acid (PGLA) and polymer surfactant Poloxamer® 188.

The patent description WO2011086093 discloses compositions for oral delivery of peptides, including insulin, with the use of self-microemulsifying drug delivery systems (SMEDDS). In order to overcome instability of the peptide in the carrier system (protection against degradation or deactivation in the acidic gastric environment) it is embedded in a coated soft capsule which, unfortunately, exhibits delayed activity after oral administration. Furthermore, the rate of gastric emptying differs from person to person, and this affects the timing of insulin release from the formulation and proper absorption by the intestines. Such changes lead to significant differences in insulin absorption, potentially leading to uncontrolled blood sugar. The problems also include the possible incompatibilities in the carrier-drug system.

The related literature does not present methods for producing and stabilizing water-in-oil-in-water double emulsions which would not require addition of small-particle or large-particle surface-active compounds or other stabilizers with an ability for concurrent efficient encapsulation of hydrophobic and hydrophilic compounds, to enable oral delivery of active substances. This issue has been achieved in the present invention.

The object of the present invention is a water-in-oil-in-water (W/O/W) emulsion system, with a nanocapsule-in-nanocapsule structure, where small-molecule surfactants, emulsifiers and/or stabilizers are not required for the system stability. The said system functions as a carrier which enables protection of sensitive hydrophilic substances against aggressive external environment, and the resulting degradation and deactivation, and makes it possible to concurrently administer active substances of varied hydrophilicity, and in particular enables delivery of proteins.

The object of the current invention is to provide novel water-in-oil-in-water emulsion systems (nanocapsule-in-nanocapsule). The new systems, being pharmaceutical dosage forms, may contain antitumor-active substances or proteins.

### DETAILED DESCRIPTION OF THE INVENTION

The object of the current invention is a biocompatible water-in-oil-in-water double emulsion system designed for concurrent delivery of lipophilic compounds (in oil phase) and hydrophilic compounds (in inner aqueous phase). Rather than by using small-particle surface-active compounds (surfactants), stability of the system is ensured by hydrophobically modified hyaluronic acid.

The produced stabilizing shell of the capsule with oil core and the capsule with aquatic core (inner capsule) consist of hydrophobically modified sodium hyaluronate, Hy-Cx, with a formula:

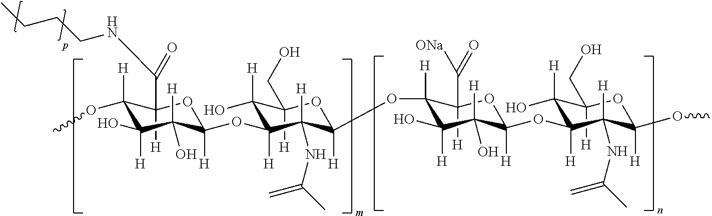

where x is an integral number in the range of 1-30 and it defines the total number of carbon atoms in the hydrophobic side chain, the ratio of the numbers m/(m+n) ranges from 0.001 to 0.4;

A nanocapsule-in-nanocapsule system is produced in a two-stage process. During the first stage inverted emulsion of water-in-oil type is produced by mixing an aqueous solution e.g. of a hyaluronic acid dodecyl derivative with a non-toxic oil constituting 80%-99.9% of the mixture volume. At the next stage, the water droplets suspended in the continuous oil phase receive hyaluronate coating, as a result of which water-in-oil-in-water double emulsion is produced. The second stage is necessary because it allows to achieve stability of the colloidal system; the W/O system produced during the first stage is unstable, while the double emulsion exhibits stability for a minimum of two months.

To obtain a W/O/W emulsion, which is stable over time, it is necessary to maintain a balance between hydrophilic and hydrophobic fragments of a polysaccharide macromolecule. It is beneficial if the degree of substitution of hydrophobic groups in the polysaccharide chain is in the range of 0.1%-40%. Research conducted showed that the best properties are exhibited by a system stabilized by hyaluronic acid modified with dodecyl side chains. The most effective degree of substitution in a polysaccharide chain does not exceed 5%. This is because excessive content of hydrophobic chains may reduce solubility of the polymer in water.

To achieve good stability of the system, it is also important to use polysaccharides containing ionic groups, e.g. carboxyl groups. It is advantageous if the contents of ionic groups in the polysaccharide is greater than 20 mol-% (calculated per one mer), it is more effective if the content is greater than 40 mol-%, and the most effective if it exceeds 60 mol-%.

It is necessary to apply sonication (or dynamic mixing) in order to obtain both W/O inverted emulsion and W/O/W double emulsion; It is advantageous if sonication is continued for 15-60 minutes, at a temperature higher than 18° C., but not exceeding 40° C. It is most effective if the sonication continues for 60 min to obtain inverted emulsion and 30 min to obtain double emulsion and if the process is carried out at a temperature in the range of 25-30° C.

Stable double emulsions are produced using aqueous solutions of hydrophobically modified ionic polysaccharides with concentrations of 0.1-20 g/L and ionic strength in the range of 0.001-1.0 mol/dm³. It is advantageous to apply a 2 g/L solution of hyaluronic acid dissolved in 0.15 mol/dm³ solution of sodium chloride

The obtained nanocapsule-in-nanocapsule systems can be used for a wide spectrum of purposes because they enable concurrent encapsulation of hydrophobic compounds (to oil phase) and hydrophilic compounds (to inner aqueous phase). It is possible to encapsulate fluorescent dyes for imaging examinations. Concurrent application of hydrophilic and hydrophobic dyes enables imaging of capsule geometry. It is also possible to use fluorescently labeled derivatives of hyaluronic acid. It is advantageous to apply dyes with varied spectral characteristics; it is more effective to use dyes excited by different lasers and emitting radiation in varied channels of emission in confocal fluorescence microscopy. It is most effective to use of hyaluronic acid modified with rhodamine isothiocyanate or fluorescein isothiocyanate.

The object of the current invention is a multicompartment system of nanocapsule-in-nanocapsule type, in a form of water-in-oil-in-water double emulsion, for concurrent delivery of hydrophilic and lipophilic compounds, which comprises:
a) liquid oil core for transport of a lipophilic compound, containing oil selected from the group including: oleic acid, isopropyl palmitate, fatty acids, natural extracts and oils, such as corn oil, linseed oil, soybean oil, argan oil, or their mixtures; beneficially oleic acid;
b) embedded in the oil core, a capsule or many capsules with aqueous core, for transport of a hydrophilic compound;
c) stabilizing shell for both the capsule with oil core and the inner capsule with water core, consisting of a hydrophobically modified polysaccharide selected from a group comprising: derivatives od chitosan, oligochitosan, dextran, carrageenan, amylose, starch, hydroxypropyl cellulose, pullulan and glycosaminoglycans, hyaluronic acid, heparin sulfate, keratan sulfate, heparan sulfate, chondroitin sulfate, dermatan sulfate; beneficially derivatives of hyaluronic acid;
d) outer capsule diameter below 1 μm, stable in aqueous solution;
e) active substance:

A system where the degree of hydrophobic side chains substitution in a hydrophobically modified polysaccharide ranges from 0.1 to 40%.

A system where stabilizing shells for the capsule with oil core and the capsule with water core (inner capsule) consist of hydrophobically modified sodium hyaluronate, Hy-Cx, with a formula:

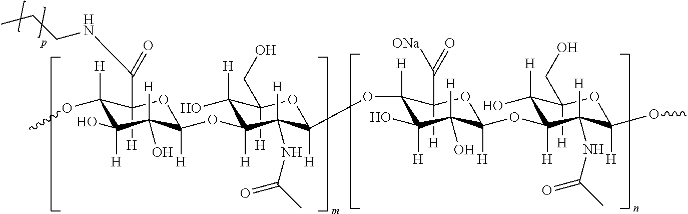

where x is an integral number in the range of 1-30 and it defines the total number of carbon atoms in the hydrophobic side chain, the ratio of the numbers m/(m+n) ranges from 0.001 to 0.4.

A system where the transported lipophilic compound may be a fluorescent dye, fat-soluble vitamin, or a hydrophobic drug.

A system where the transported hydrophilic compound may be a fluorescent dye, water-soluble vitamin, protein or a hydrophilic drug; advantageously: insulin.

A system where insulin is in a concentration of 0.005-20.000 of insulin units per 1 ml of the capsule suspension.

A method of producing a multicompartment system of nanocapsule-in-nanocapsule type, in a form of water-in-oil-in-water double emulsion, as defined in claim 1, where:
a) during the first step inverted emulsion of water-in-oil (W/O) type is produced by mixing an aqueous solution of hyaluronic acid dodecyl derivative Hy-Cx, described by the above formula, with a non-toxic oil constituting about 0.1-99.9% of the mixture volume, by exposition to ultrasounds (sonication) or to mechanical stimuli, advantageously—mixing or shaking, with aqueous phase to oil phase volume ratio ranging from 1:10 to 1:10000; advantageously approx. 1:100;
b) during the second step, water droplets suspended in the continuous oil phase receive hyaluronate coating, with W/O phase emulsion to water phase volume ratio ranging from 1:10 to 1:10000; advantageously approx. 1:100,
c) as a result, water-in-oil-in-water (W/O/W) double emulsion system is produced by exposition to ultrasounds (sonication) or to mechanical stimuli, advantageously—mixing or shaking,
   wherein, the water phase applied is based on aqueous solution of hydrophobically modified polysaccharide selected from a group comprising: derivatives of chitosan, oligochitosan, dextran, carrageenan, amylose, starch, hydroxypropyl cellulose, pullulan and glycosaminoglycans, and particularly hyaluronic acid, heparin sulfate, keratan sulfate, heparan sulfate, chondroitin sulfate, dermatan sulfate; advantageously derivatives of hyaluronic acid with pH in the range of 2-12, concentration of 0.1-30 g/L and ionic strength in the range of 0.001-3 mol/dm³,
   and the oil phase contains oil selected from the group including: oleic acid, isopropyl palmitate, fatty acids, natural oils, in particular linseed oil, soybean oil, argan oil, or their mixtures; beneficially oleic acid,
   notably, the process is carried out without using any small-particle surfactants.

A method where pulse sonication is carried out with impulse duration twice as short as the duration of the interval between two consecutive impulses.

A method where the encapsulated lipophilic compound is contained in the oil core and the encapsulated hydrophilic compound is comprized in the water core of the nanocapsule.

A method where it is advantageous if the content of ionic groups in the polysaccharide is not lower than 20 mol %, and advantageous if it exceeds 60 mol-% (calculated per one mer).

A method where during the first and second step, sonication is continued for 15-60 minutes, at a temperature of 18° C.-40° C., advantageously for 60 min to obtain inverted emulsion and 30 min to obtain double emulsion, at a temperature of 25-30° C.

Application of the multicompartment system, as defined above, for transport of lipophilic compounds and hydrophilic compounds, where the lipophilic compound may be a fluorescent dye, fat-soluble vitamin, or a hydrophobic drug, while the hydrophilic compound may be a fluorescent dye, water-soluble vitamin, protein or a hydrophilic drug; advantageously: insulin.

The advantages of the said invention include the possibility to obtain a biocompatible and stable nanoformulation able to concurrently deliver hydrophilic and lipophilic compounds in separate compartments of a double nanocapsule. This protects the encapsulated compounds against degradation, untimely release from the carrier, and excessively rapid elimination from the system, e.g. blood circulation. This significantly improves the range of applications of the said systems which are also characterized by simplicity of preparation and low financial costs. Furthermore, the use of the carrier system enables oral administration of peptides and other active substances as well as improvement of their bioavailability.

### DESCRIPTION OF THE TABLES AND FIGURES

The object of the invention is shown in the examples and figures, listed below:

FIG. 1—presents the inverted emulsion obtained by mixing a pre-emulsion containing water and oleic acid, with water-ethanol solution of hyaluronic acid dodecyl derivative (water:alcohol volume ratio of 2:3) described in Example I. The arrows indicate large bubbles created during emulsification.

FIG. 2—presents bubbles created during the process of producing the inverted emulsion which was obtained by mixing a pre-emulsion containing water and oleic acid, with water-ethanol solution of hyaluronic acid dodecyl derivative (water:alcohol volume ratio of 1:2) described in Example II.

FIG. 3—presents the inverted emulsion described in Example III, obtained by mixing a pre-emulsion containing water and oleic acid, with water solution of hyaluronic acid dodecyl derivative, one day (a) and five days (b) after it was produced.

FIG. 4—presents molecule-size distribution in the inverted emulsion described in Example III, obtained by mixing a pre-emulsion containing water and oleic acid, with water solution of hyaluronic acid dodecyl derivative (configuration on the day of emulsification).

FIG. 5—presents molecule-size distribution in the inverted emulsion described in Example III, obtained by mixing a pre-emulsion, containing water and oleic acid, with water solution of hyaluronic acid dodecyl derivative (5 days after emulsification).

FIG. 6—presents a cryo-TEM microphotograph of a molecule of the inverted emulsion (W/O) described in Example IV, obtained by mixing a pre-emulsion, containing water and oleic acid, with water solution of hyaluronic acid dodecyl derivative containing sodium tungstate (VI).

FIG. 7—presents molecule-size distribution in the double emulsion described in Example V, obtained by mixing 0.4 vol. % of inverted emulsion containing FITC labeled hyaluronic acid dodecyl derivative with water solution of RhBITC-labeled hyaluronate (configuration on the day of emulsification).

FIG. 8—presents molecule-size distribution in the double emulsion described in Example V, obtained by mixing 0.4 vol. % of inverted emulsion containing FITC labeled hyaluronic acid dodecyl derivative with water solution of RhBITC-labeled hyaluronate (configuration 7 days after emulsification).

FIG. 9 presents confocal microscopy images of the double emulsion system described in Example VI, obtained by mixing 0.4 vol. % of inverted emulsion containing FITC labeled hyaluronic acid dodecyl derivative with water solution of RhBITC-labeled hyaluronate—observation in the cumulative channel (a) and in FITC channel (b) (5 μm scale).

FIG. 10 presents a cryo-TEM microphotograph of a molecule of the double emulsion described in Example VII, obtained by mixing 0.4 vol. % of inverted emulsion containing FITC labeled hyaluronic acid dodecyl derivative and dissolved sodium tungstate (VI) with water solution of RhBITC-labeled hyaluronate.

FIG. 11 presents molecule-size distribution in the double emulsion described in Example VIII, containing calcein in the inner aqueous phase.

FIG. 12 presents confocal microscopy images of the double emulsion system described in Example VIII—observation in the cumulative/collective channel—overlapping of the signal from calcein and rhodamine which was used to modify hyaluronate (10 μm scale).

FIG. 13 presents molecule-size distribution in the double emulsion described in Example IX, obtained by mixing 0.1 vol. % of inverted emulsion containing FITC labeled hyaluronic acid dodecyl derivative (aqueous-oil phase volume ratio of 1:30) with water solution of RhBITC-labeled hyaluronate.

FIG. 14 presents confocal microscopy images of the double emulsion described in Example IX, obtained by mixing 0.1 vol. % of inverted emulsion containing FITC labeled hyaluronic acid dodecyl derivative (aqueous-oil phase volume ratio of 1:30) with water solution of RhBITC-labeled hyaluronate. Observation in the cumulative channel (a), FITC channel (b) and TRITC channel (c) (10 μm scale).

FIG. 15 presents molecule-size distribution in the double emulsion described in Example X, eleven weeks after W/O/W system was produced.

FIG. 16 presents a listing of zeta potentials and standard deviations (SD) of the W/O/W system described in Example X, measured on the day the double emulsion system was obtained as well as following 7, 14, 21, 28, 43, 59 and 79 days.

FIG. 17 presents confocal microscopy images of the double emulsion system described in Example X—observation in the cumulative channel, after week 3 (top panel), and after week 4 (bottom panel) (5 μm scale).

FIG. 18 presents molecule-size distribution in the double emulsion described in Example XI, containing calcein in the inner aqueous phase and Nile red in the oil phase.

FIG. 19 presents images of double emulsion system described in Example XI, containing calcein in the aqueous phase and Nile red in the oil phase, obtained with confocal microscope—observation in TRITC channel (a, Nile red), FITC (b, calcein) and in cumulative channel (c) (5 μm scale).

FIG. 20—presents nanocapsule-size distribution of the double emulsion described in Example XII, on the day (a), one week (b) and two weeks (c) after double emulsion was produced following the procedure described in example 1.

FIG. 21—presents a photograph showing a small outflow of the oil phase to the surface and dilution of the emulsion described in Example XII, one week after double emulsion was produced following the procedure described in example 1.

FIG. 22—presents a photograph showing a small outflow of the oil phase to the surface and dilution of the emulsion described in Example XII, two weeks after double emulsion was produced following the procedure described in example 1.

FIG. 23—presents confocal microscopy images of the capsules described in Example XII on the day they were prepared, using measurements in transmitted light mode (a) and using TRITC filter (b)—images collected using a confocal microscope.

FIG. 24—presents nanocapsule-size distribution on the day double emulsion described in Example XIII was produced (a), one week (b), two weeks (c) and three weeks (d) after the double emulsion was produced following the procedure described in example 2.

FIG. 25—presents confocal microscopy images of the capsules described in Example XIII on the day they were prepared, using measurements in transmitted light mode (a, c) and using TRITC filter (b, d)—images collected using a confocal microscope.

FIG. 26—presents confocal microscopy images of the capsules described in Example XIII, three weeks after they were produced, using measurements in transmitted light mode (a) and using TRITC filter (b))—images collected using a confocal microscope.

FIG. 27—presents nanocapsule-size distribution of the double emulsion described in Example XIV on the day (a), and one week (b) after the double emulsion was produced following the procedure described in example 3.

FIG. 28—presents confocal microscopy images of the capsules described in example XIV on the day they were produced, using measurements in transmitted light mode (a) and using TRITC filter (b))—images collected using a confocal microscope.

FIG. 29—presents nanocapsule-size distribution of the double emulsion described in Example XV, on the day (a), and one week (b) after double emulsion was produced.

FIG. 30—presents nanocapsule-size distribution of the double emulsion described in Example XVI, on the day (a), and one week (b) after double emulsion was produced.

FIG. 31—presents results of glucose level measurements described in Example XVII, in group 1 and 2 (a) as well as 3, 4 and 5 (b) calculated as a mean value, with reference to the relevant control group.

The invention is illustrated by the following non-limiting examples

### EXAMPLE I

### Method of Making Inverted Emulsion of Water-in-Oil Type

In order to produce inverted emulsion (W-O type), water-ethanol solution of hyaluronic acid dodecyl derivative was applied. The presence of the volatile organic solvent was to enable polymer chains to achieve extended conformation (to produce the inverted emulsion). The solvent subsequently was to be evaporated.

Solution of hyaluronic acid dodecyl derivative (degree of hydrophobic side chains substitution from 4.5%) was prepared in physiological saline (concentration approx. 7.5 g/L). The neutral solution was then ethanolized and a mixture with 2:3 volume ratio was obtained.

Concurrently a pre-emulsion was prepared by mixing oleic acid with aqueous solution of sodium chloride (c=0.15 mol/dm₃), at volume ratio of 100:1. The system was subjected to shaking for 10 minutes in a vortex type shaker, and subjected to sonication for 30 minutes in an ultrasonic cleaner (pulsed mode, 1 s ultrasounds, 2 s interval) in room temperature. As a result of sonication, a milk-white emulsion was produced.

Water-ethanol solution of hyaluronic acid dodecyl derivative was gradually added drop by drop to the pre-emulsion, for 5 minutes. The whole mixture was subjected to sonication for 30 min in pulsed mode, in an open bottle, in order to evaporate the ethanol.

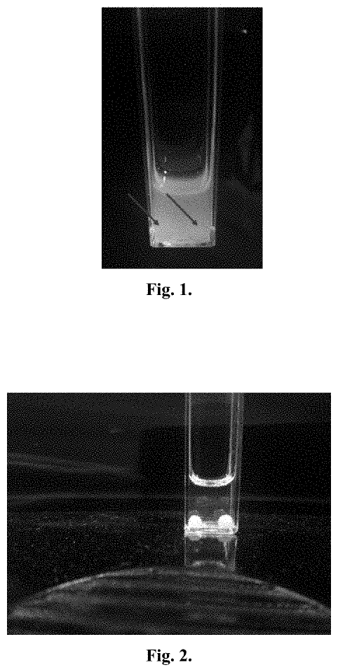

Size distributions measured using dynamic light scattering (DLS) show that the system contained many molecular fractions. It was impossible to measure zeta potential (ξ) indicating stability of the system (highly unstable measurements). Furthermore, the bottle contained visible spherical bubbles with diameters exceeding 1 mm (FIG. 1).

### EXAMPLE II

### Method of Making Inverted Emulsion of Water-in-Oil Type, after Decreasing the Content of Aqueous Phase in the Water-Ethanol Solution

Pre-emulsion was prepared as described in Example I. Water-ethanol solution of hyaluronic acid dodecyl derivative was added gradually, however aqueous phase to ethanol phase volume ratio of 1:2 was applied.

In order to evaporate the ethanol, the system was subjected to sonication at a higher temperature (about 34° C.).

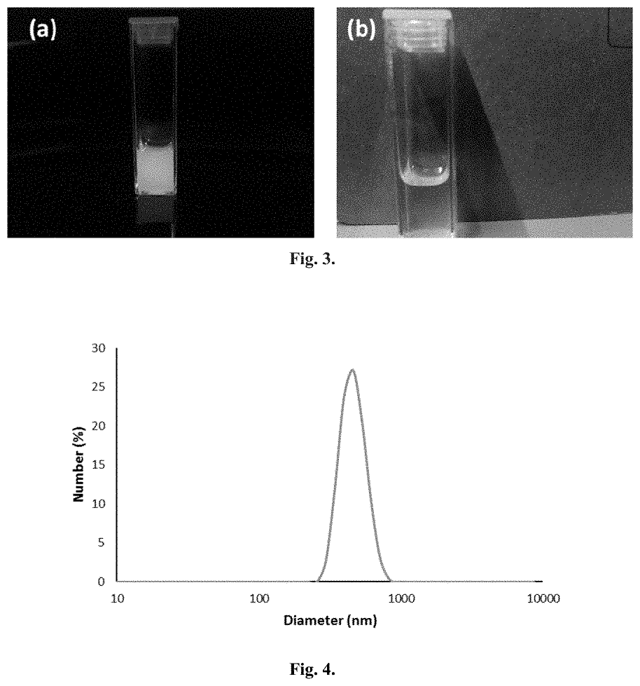

Initially white suspension could be seen in the oil; after the system was introduced into the cuvette used in DLS measurements, the suspension transformed into bubbles with diameters exceeding 1 mm (FIG. 2).

After the sizes were measured in DLS apparatus, 2 large water drops were observed in the cuvette. Zeta potential could not be measured

Based on the results presented in Examples I and II, it was concluded that ethanol adversely affected production of the emulsion; at the next step alcohol was eliminated from the system.

### EXAMPLE III

### Method of Making Inverted Emulsion of Water-in-Oil Type, after Eliminating Alcohol from the System

Inverted emulsion of water-in-oil type was prepared by mixing a solution of hyaluronic acid dodecyl derivative (c=4.7 g/L) in physiological saline (c_{NaCl}=0.15 mol/dm³) with oleic acid, at a volume ratio of 1:100. The system was subjected to shaking and sonication, as described in Example I, however sonication process continued for one hour.

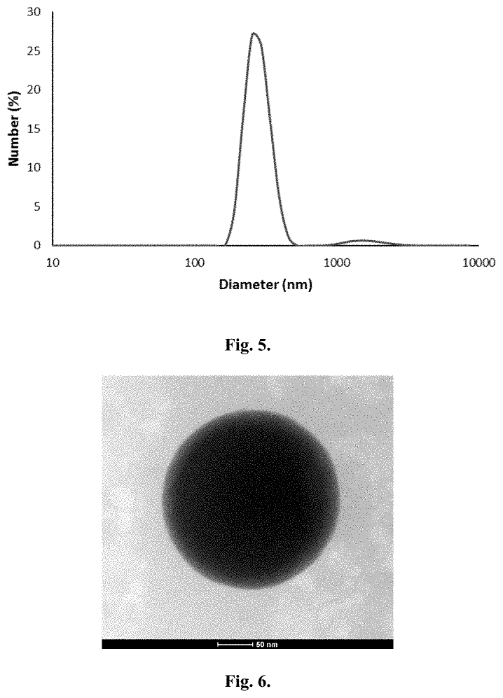

A milk-white emulsion was obtained, and its stability was measured on the day and five days after the emulsification. The DLS tests showed high stability of the initial system (ξ=−33±21.7 mV). The molecular sizes were characterized by narrow distribution. After five days, the distribution describing molecule sizes shifted towards smaller molecules; additionally, another small maximum could be observed. After five days there was a significant decrease in the turbidity of the sample (FIG. 3, FIG. 4, FIG. 5). Visual observation combined with DLS data enabled a conclusion that after five days there was a decrease in the contents of molecules, which suggests that the obtained system comprised both stable and unstable elements. From the viewpoint of applicability, this situation poses a disadvantage because it leads to loss of material and to production of a system with uncontrolled composition. Due to the above, at the next stage the inverted emulsion system was directly subjected to the subsequent steps leading to production of a double emulsion.

### EXAMPLE IV

### Inverted Emulsion Imaging with Cryoscopic Transmission Electron Microscopy

Inverted emulsion was prepared following the procedure described in Example III, however the inner aqueous phase contained sodium tungstate (VI), in order to enhance contrast during the imaging examination. Two days later the emulsion was examined using transmission electron microscopy technique, supplemented with cryoscopy device. Analysis of the acquired images confirms presence of spherical molecules with a diameter of approx. 250 nm (FIG. 6).

### EXAMPLE V

### Method of Making Double Emulsion

Inverted emulsion was prepared as in Example III, however dodecyl derivative of fluorescein isothiocyanate (FITC) labeled hyaluronic acid was applied at a concentration of 2 g/L, and sonication continued for 30 minutes.

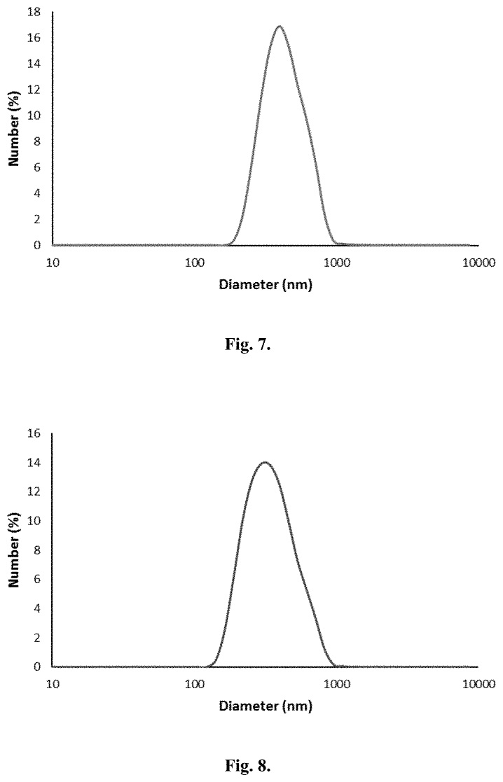

Double emulsion was obtained by mixing inverted emulsion constituting 0.4% volume of the mixture with dodecyl derivative of rhodamine isothiocyanate (RhBITC) labeled hyaluronic acid at a concentration of 1 g/L in physiological saline. The system was subjected to shaking for 10 minutes in a vortex type shaker, and subjected to sonication in room temperature for 30 minutes, in accordance with the parameters described in Example I. Analysis of molecule-size distributions in DLS tests shows there are molecules with diameters of 500-600 nm, while zeta potential measurement confirms stability of the obtained system (ξ=−44.6±3.33 mV). After seven days of observations no significant changes were shown in molecule sizes or the value of zeta potential (ξ=−44.6±3.08 mV) (FIG. 7, FIG. 8).

### EXAMPLE VI

### Double Emulsion Imaging with Confocal Microscopy

Labeled polysaccharides were applied to visualize the structures obtained in Example V, using confocal microscopy. Because of the spectral characteristics both dyes can be excited with lasers of varied wavelength (488 nm and 561 nm), and emissions can be observed in other microscope channels. It was shown that FITC is not excited by the laser corresponding to RhB (and vice versa); RhB signal was not observed in FITC channel, and FITC signal was not identified in the channel corresponding to rhodamine emission.

By applying the derivative containing FITC in the first W-O type emulsion, and the derivative containing RhBITC at the second stage to produce double emulsion, it was possible to visualize the obtained structures and confirm their morphology.

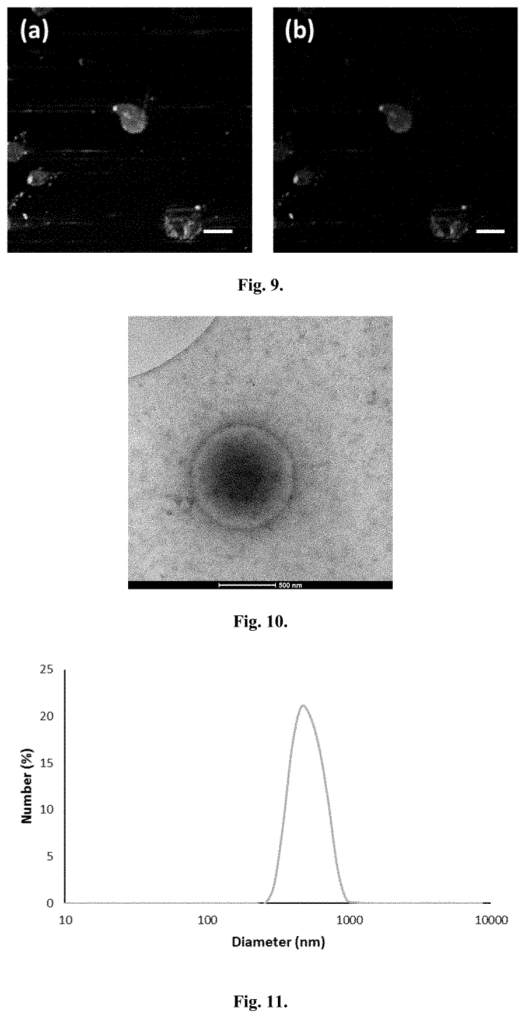

Images from confocal microscope (100× lens, 488 nm and 561 nm lasers) confirm presence of a “layered” sheath—observation of signal from all the channels and the channel characteristic for FITC (FIG. 9).

### EXAMPLE VII

### Double Emulsion Imaging with Cryoscopic Transmission Electron Microscopy

Double emulsion was prepared following the procedure described in Example V, however the inner aqueous phase contained sodium tungstate (VI), in order to enhance contrast during the imaging examination. After two days a sample was examined using transmission electron microscopy technique, and cryoscopy device. Analysis of the acquired images confirms presence of spherical molecules with a diameter of approx. 600 nm (FIG. 10)

### EXAMPLE VIII

### Encapsulation of Hydrophilic Dye in the Inner Aqueous Phase

Double emulsion was prepared as described in Example V, however inverted emulsion was prepared from water solution of hyaluronic acid dodecyl derivative with concentration of 4.5 g/L in physiological saline mixed with calcein solution (c_{kalc}=2 g/L) at 3:1 volume ratio. Analysis of molecule sizes based on results of DLS measurements confirmed the formulation obtained was stable (ξ=−32.5±6.58 mV) and contained molecules with hydrodynamic diameters of approx. 600 nm (FIG. 11). The findings showed no effects of the encapsulated substance in the physicochemical properties of the colloidal system.

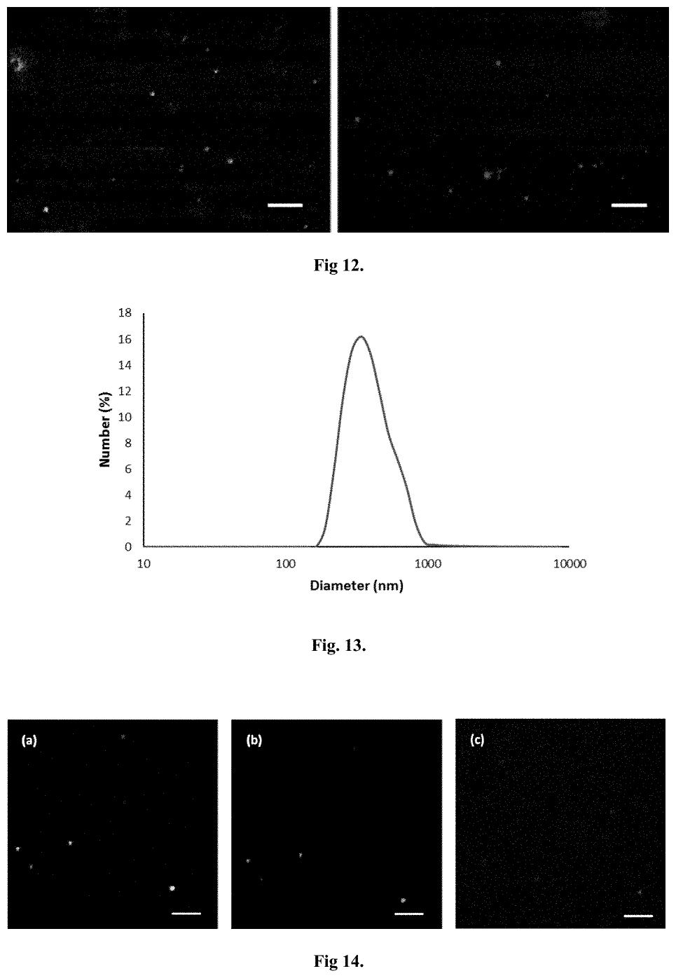

Confocal microscopy images (observation in all the channels) confirm that a nanocapsule-in-nanocapsule system was obtained, which is shown by a signal visible in both channels, and overlapping within the molecules observed (FIG. 12)

### PRZYKŁAD IX

### Optimization of Double Emulsion Composition

In order to optimize the sizes and composition of the obtained system, a change was introduced in the volume ratio of aqueous and oil phase in the inverted emulsion, which was made as described in Example VIII, with aqueous phase to oil phase volume ratio of 30:1. Double emulsion was obtained by mixing the inverted emulsion and dodecyl derivative of rhodamine isothiocyanate labeled hyaluronic acid with a concentration of 1 g/L. The content of the inverted emulsion in the mixture amounted to 0.1% volume. Sonication was conducted as described in Example V. The obtained system was characterized by narrow distribution of molecule sizes (FIG. 13), with high stability measured by the value of zeta potential (ξ=−31.0±2.32 mV). Observation via confocal microscope (100× lens, 488 nm and 561 nm lasers) confirmed that a nanocapsule-in-nanocapsule system was formed (FIG. 14).

### EXAMPLE X

### Long-Term Stability of Double Emulsion

Stability of the water-in-oil-in-water double emulsion produced using hyaluronic acid dodecyl derivative was tested over a period of 11 weeks. The parameters of the system were examined in specified points of time using dynamic light scattering technique and confocal microscopy. The capsules were produced as described in Example IX.

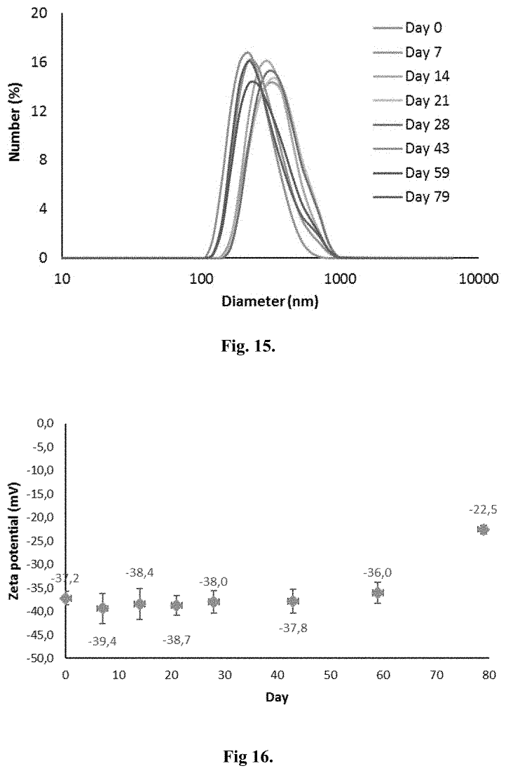

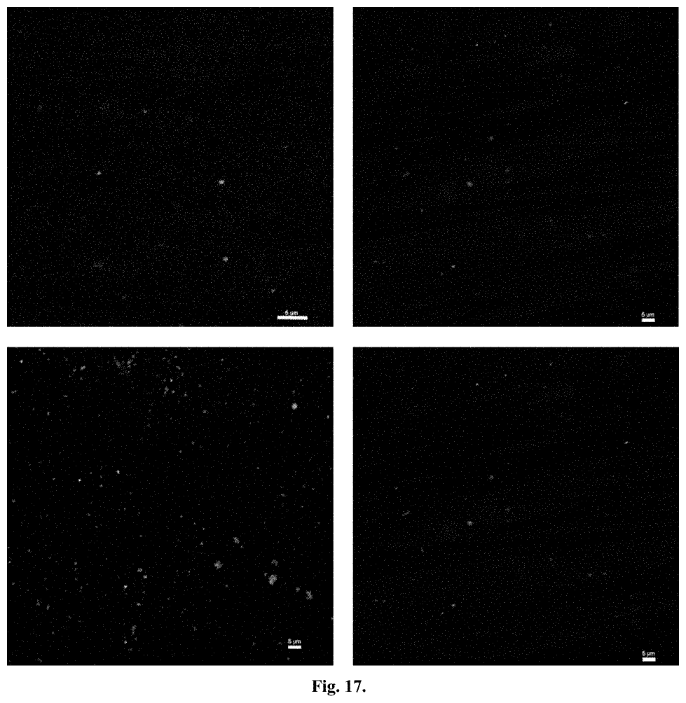

The obtained system was characterized by monomodal molecule size distribution (FIG. 15), with high stability measured by the value of zeta potential (ξ=−37.2±1.4 mV) (FIG. 16). During the tests assessing the stability of the system, the maximum of size distribution was slightly shifted towards larger molecules. The stability defined by the measure of zeta potential in the system did not deteriorate after 11 weeks of observations. Observation of the system via confocal microscope confirmed that a “nanocapsule-in-nanocapsule” system was formed (overlapping signal from both fluorescence channels) (FIG. 17).

### EXAMPLE XI

### Preparation and Visualization of Double Emulsion Containing Dissolved Fluorescent Dyes

Inverted emulsion was made by mixing oleic acid with solution of hyaluronic acid dodecyl derivative, in physiological saline, as described in Example IX, with Nile Red dye dissolved in the oil phase (c=0.85 g/L), and calcein dissolved in the aqueous phase (c=0.17 g/L). Double emulsion was produced as described in Example IX.

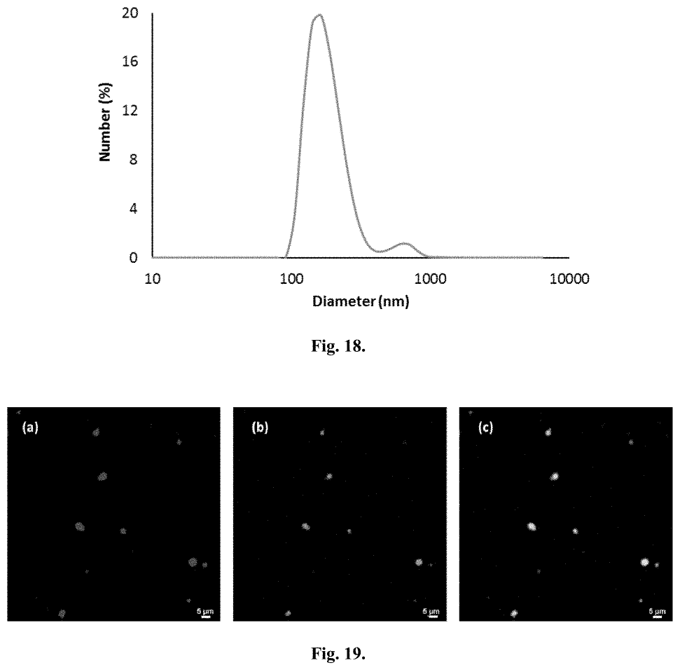

The obtained molecules were characterized by hydrodynamic diameter similar to that in the molecules formed in Example X (FIG. 18). The size distribution contains a visible proportion of molecules with a diameter of approx. 700 nm.

Visualization performed using confocal microscope showed that a nanocapsule-in-nanocapsule system was formed (overlapping signal from both fluorescence channels) (FIG. 19).

### EXAMPLE XII

### 1) Preparation of Insulin Solution:

21.66 mg of insulin (Sigma Aldrich) was dissolved in 1 ml 0.15M NaCl (addition of 4 μl 3M HCl, pH ˜1.9), i.e. approx. 600 UI/ml (3.56 mg=100 UI)*.

The process produced clear insulin solution which retained the lucid form when stored at a temperature of 4° C. (two-week observations).

Subsequently, insulin solution was prepared with an addition of a dye, i.e. Neutral Red (C=1 g/l in 0.15M NaCl) (180 μl insulin solution+20 μl dye solution).

No negative effect of the dye added to insulin solution was observed.

### 2) Preparation of Capsules

a) Emulsion 1:

In accordance with the procedures described above in this invention, Emulsion 1 was obtained following the formula: 3.6 ml of oleic acid was emulsified with 100 μl of HyC12 solution (C=4.6 g/l in 0.15M NaCl) and 20 μl of insulin solution with a dye; the process was carried using Vortex-type shaker (10 min) and ultrasounds (pulsed mode, 30 min).

b) Emulsion 2:

Emulsion 2 was made from 6 ml of HyC12 solution (C=1 g/l in 0.15M NaCl) and 12 μl of Emulsion 1. The mixture was emulsified using Vortex shaker (10 min) and ultrasounds (30 min, pulsed mode).

Milk-white emulsion was obtained.

1 ml of the capsules contained 0.01 μl of insulin solution, i.e. 0.0061 units of insulin per 1 ml of the capsules.

### 3) Characteristics:

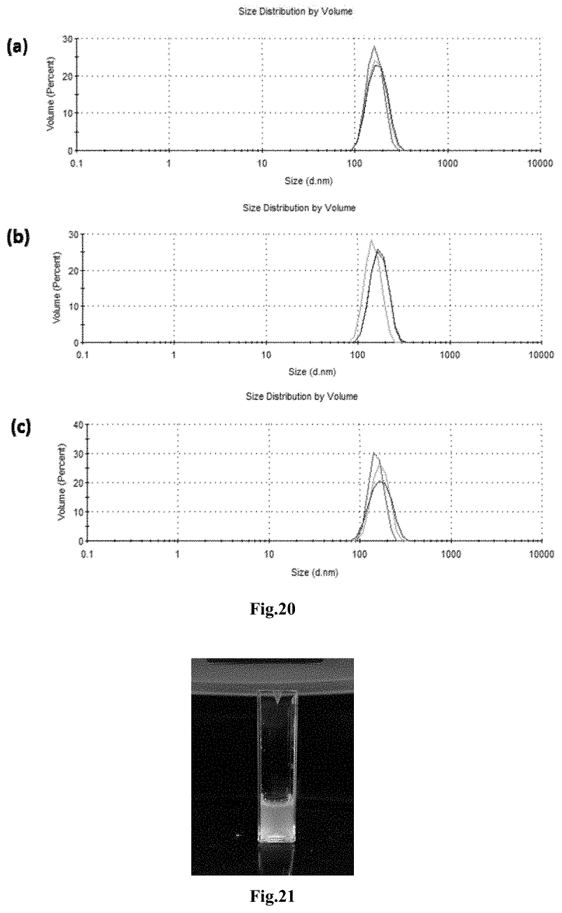

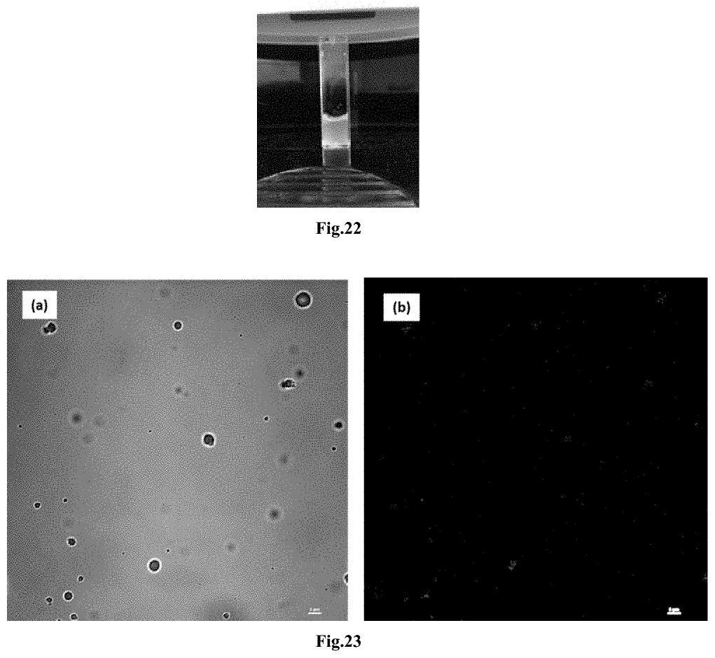

The obtained W/O/W emulsion consisted of suspended molecules with hydrodynamic diameter of up to 180 nm. It was highly stable, as shown by the high value of zeta potential. The capsules were stored at a temperature of 4° C. After one week a small outflow of the oil phase to the surface was observed along with dilution of the emulsion. Measurements performed using dynamic light scattering (DLS) technique showed a slightly reduced modular value of zeta potential and a decrease in the molecule sizes. The results are presented in Table 1 and in FIG. 20-23.

TABLE 1
Summary measures of hydrodynamic diameters (volume means)
and zeta potentials in the W/O/W system, on the day as well as one
and two weeks after the emulsion was produced.

| | dv [nm] | Zeta potential [mV] |
|---|---|---|
| Time [week] | [Diss. 100x] | [Diss. 100x] |
| 0 | 173 ± 6 | −45 ± 3 |
| 1 | 165 ± 14 | −37 ± 1 |
| 2 | 165 ± 11 | −38 ± 4 |

### EXAMPLE XIII

### 1) Preparation of Insulin Solution.

The insulin solution from Example 1 was condensed with additional solution of 49.73 mg of insulin, and acidified with an addition of 6 μl of muriatic acid (C=3 mol/dm³) in order to obtain a clear solution, which was then subjected to shaking in Vortex shaker for 5 min

The obtained insulin had a concentration of 81.34 mg/ml (2284.75 UI).

The first component of Emulsion 1 was prepared by mixing 30 μl of HyC12 solution (C=15 g/l in 0.15M NaCl) with 80 μl of insulin solution and 10 μl of the dye (Neutral Red, C=3.5 mg/ml in 0.15M NaCl).

### Emulsion 1:

A mixture of 120 μl of the first component of Emulsion 1 and 3.6 ml of oleic acid was subjected to shaking in Vortex shaker for 10 min, and then to sonication in pulsed mode, for 30 min

### Emulsion 2:

A mixture of 20 μl of Emulsion 1 and 2 ml of HyC12 solution (C=5 mg/ml in 0.15M NaCl) was subjected to shaking in Vortex shaker for 10 min, and then to sonication in pulsed mode, for 30 min. The obtained milky, viscous and very dense emulsion contained 0.49 units of insulin per 1 ml.

### Characteristics:

The obtained capsules were characterized by good stability, reflected by the high values of zeta potentials. The encapsulated dye also influenced these high values. The capsules were stored at a temperature of 4° C. After one and two weeks the emulsion retained its stability. Following one week (and later) measurements of hydrodynamic diameters, high dispersion indicator, and confocal microscopy show that aggregates and larger structures are formed, and there is no evidence of monodispersity in the sample.

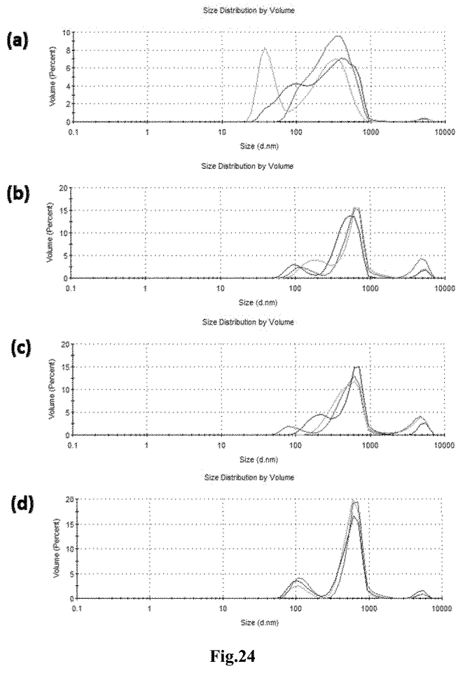

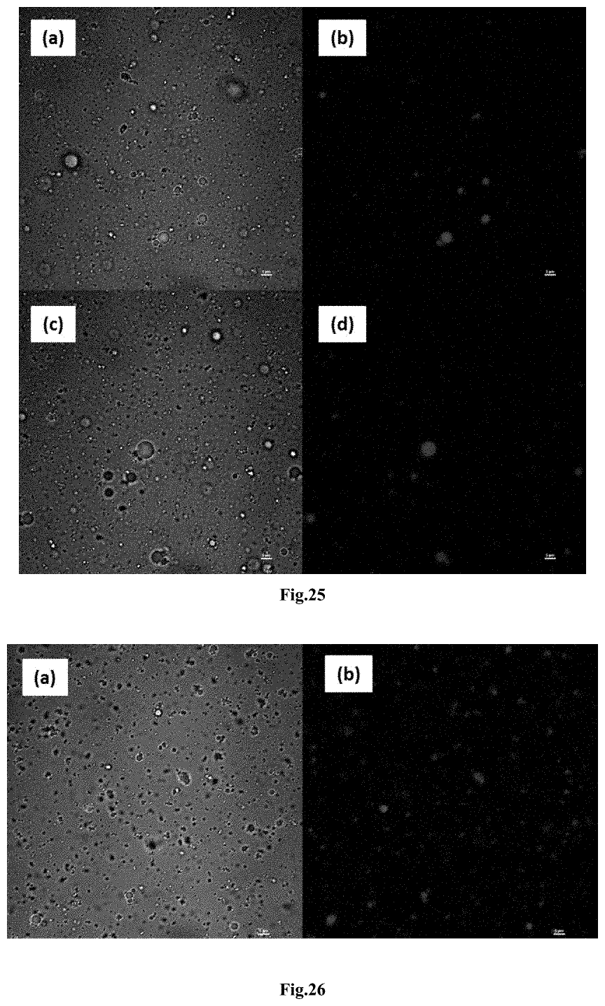

For the purpose of the measurements the capsules were diluted (100×) with 0.15M NaCl solution. The results are shown in Table 2 and FIG. 24-26.

TABLE 2
Summary measures of hydrodynamic diameters (volume means)
and zeta potentials in the W/O/W system, on the day as well as
one, two and three weeks after the emulsion was produced.

| | dv [nm] | Zeta potential [mV] |
|---|---|---|
| Time [week] | [Diss. 100x] | [Diss. 100x] |
| Day 1 | 313 ± 51 | −59 ± 0 |
| 1 | 883 ± 265 | −53 ± 2 |
| 2 | 1062 ± 178 | −51 ± 3 |
| 3 | 668 ± 40 | −48 ± 2 |

### EXAMPLE XIV

### Emulsion 1: Produced Following the Procedure Described in Example 2

### Emulsion 2:

10 μl of Emulsion 1 and 2 ml HyC12 (C=2.5 mg/ml; 0.15M NaCl) were subjected to shaking in Vortex shaker for 10 min and then to sonication in pulsed mode for 30 min.

The obtained milky and viscous emulsion contained 0.245 units of insulin per 1 ml.

### Characteristics:

The obtained capsules were characterized by good stability, shown by the high values of zeta potentials. The encapsulated dye also influenced these high values. The capsules were stored at a temperature of 4° C.

After one week the emulsion retained its stability. The low PDI values reflect monodispersity of the samples and a lack of tendency for aggregation.

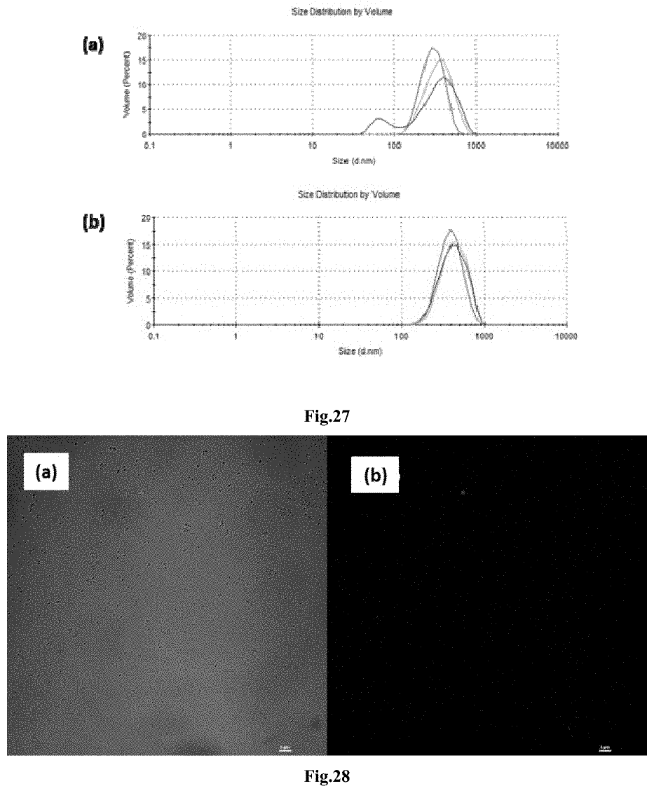

For the purpose of the measurements the capsules were diluted (100×) with 0.15M NaCl solution. The results are listed in Table 3 and FIG. 27-28.

TABLE 3
Summary measures of hydrodynamic diameters (volume means)
and zeta potentials in the W/O/W system, on the day and one week
after the emulsion was produced.

| | dv [nm] | Zeta potential [mV] |
|---|---|---|
| Time [week] | [Diss. 100x] | [Diss. 100x] |
| Day 1 | 339 ± 32 | −51 ± 2 |
| 1 | 437 ± 26 | −43 ± 2 |

### EXAMPLE XV

Preparation of insulin solution: following the procedure described in Example 2.

The first component of Emulsion 1 was prepared by mixing 60 μl of HyC12 solution (C=7.5 mg/ml in 0.15M NaCl) with 50 μl of insulin solution and 10 μl of the dye (Neutral Red C=3.5 mg/ml in 0.15M NaCl).

### Emulsion 1:

A mixture of 120 μl of the first component of Emulsion 1 and 3.6 ml of oleic acid was subjected to shaking in Vortex shaker for 10 min, and then to sonication in pulsed mode, for 30 min

### Emulsion 2:

A mixture of 10 μl of Emulsion 1 and 2 ml of HyC12 solution (C=2.5 mg/ml in 0.15M NaCl) was subjected to shaking in Vortex shaker for 10 min, and then to sonication in pulsed mode, for 30 min. The obtained milky, viscous and very dense emulsion contained 0.154 units of insulin per 1 ml.

### Characteristics:

The obtained capsules were characterized by good stability, reflected by the high values of zeta potentials. The encapsulated dye also influenced these high values. The capsules were stored at a temperature of 4° C.

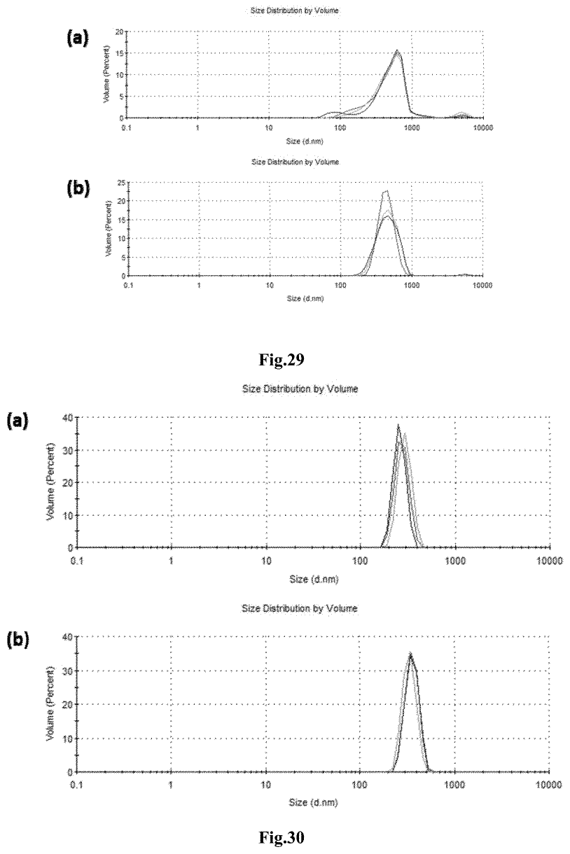

After one week the emulsion retained its stability. The obtained distributions of hydrodynamic diameters show that initially there were aggregates which disintegrated after one week. For the purpose of the measurements the capsules were diluted (100×) with 0.15M NaCl solution. The results are shown in Table 4 and FIG. 29.

TABLE 4
Summary measures of hydrodynamic diameters (volume means)
and zeta potentials in the W/O/W system, on the day
and one week after the emulsion was produced.

| | dv [nm] | Zeta potential [mV] |
|---|---|---|
| Time [week] | [Diss. 100x] | [Diss. 100x] |
| Day 1 | 615 ± 66 | −50 ± 1 |
| 1 | 476 ± 28 | −45 ± 2 |

### EXAMPLE XVI

### 1) Preparation of Insulin Solution.

The insulin solution obtained in Example 4 was condensed by adding 94 mg of insulin, and acidified with 4 μl 3M of muriatic acid in order to obtain a clear solution, which was subsequently subjected to shaking in Vortex shaker for 5 min.

The obtained insulin solution had a concentration of 200 mg/ml (5617.98 UI).

The first component of Emulsion 1 was prepared by mixing 20 μl of HyC12 solution (C=7.5 mg/ml; 0.15M NaCl) with 100 μl of insulin solution

### Emulsion 1:

A mixture of 120 μl of the first component of Emulsion 1 and 3.6 ml of oleic acid was subjected to shaking in Vortex shaker for 10 min, and then to sonication in pulsed mode, for 30 min.

### Emulsion 2:

A mixture of 10 μl of Emulsion 1 and 1 ml of HyC12 solution (C=1.5 mg/ml in 0.15M NaCl) was subjected to shaking in Vortex shaker for 20 min, and then to sonication in pulsed mode, for 35 min.

The obtained milky, viscous and very dense emulsion contained 1.5 units of insulin per 1 ml.

### Characteristics:

The obtained capsules were characterized by good stability, which was shown by the high values of zeta potentials. The capsules were stored at a temperature of 4° C. After one week the emulsion retained its stability. The distribution of hydrodynamic diameter sizes is narrow.

For the purpose of the measurements, the capsules were diluted (100×) with 0.15M NaCl solution. The results are presented in Table 5 and FIG. 30.

TABLE 5
Summary measures of hydrodynamic diameters (volume means)
and zeta potentials in the W/O/W system, on the day
and one week after the emulsion was produced.

| | dv [nm] | Zeta potential [mV] |
|---|---|---|
| Time [week] | [Diss. 100x] | [Diss. 100x] |
| Day 1 | 276 ± 17 | −39 ± 3 |
| 1 | 350 ± 13 | −46 ± 4 |

*3.56 mg=100 UI [© 2011, “Drug Discovery and Evaluation: Methods in Clinical Pharmacology”, Editors: Vogel, H. Gerhard, Maas, Jochen, Gebauer, Alexander]

### EXAMPLE XVII

### Inducing Type 1 Diabetes

A group of 30 male Wistar rats, ranging in mass from 180 to 200 g, were anesthetized with thiopental (50 mg/kg of body mass); subsequently streptozotocin (STZ) dissolved in phosphate buffer was injected via tail vein, at the rate of 60 mg/kg of body mass. The final volume of the injected solution amounted to 1 ml/kg of body mass. Blood glucose was measured three days after streptozotocin injection. Each of the animals was found with blood glucose level exceeding 450 mg % which reflected the fact that insulin-producing β cells in the pancreas were damaged. During this time the animals had unlimited access to fodder and water.

### Assessment of Encapsulated Insulin Activity

Twelve hours before the glucose tolerance test, the rats were divided into five groups of six animals (a total of 30 animals), with fodder no longer available. The animals continued to have unlimited access to water. The experiment was conducted in the following groups:
1. Control group: 2 g of glucose per 1 kg of body mass, administered via a gastric tube.
2. Insulin group: 7.5 units per 1 kilogram and 2 g of glucose per kg of body mass, administered concurrently via a gastric tube.
3. Control group: 0.5 g of glucose per 1 kg of body mass, administered via a gastric tube.
4. Insulin group: 11.25 units per one kilogram delivered 20 minutes prior to the administration of 0.5 g of glucose per 1 kg of body mass via a gastric tube.
5. Insulin group: 11.25 units per 1 kilogram and 0.5 g of glucose per kg of body mass, administered concurrently via a gastric tube.

Insulin was administered in an encapsulated form in W/O/W system obtained following the procedure described in Example 5.

In each group glucose levels were measured in blood samples collected from tail veins, at the following points of time: 0; 15; 30; 45; 60; 75; 90; 105; 120 (and 135 in groups 1 and 2). Glucose measurements were conducted using Bionime Rightest® GM100 glucose meter.

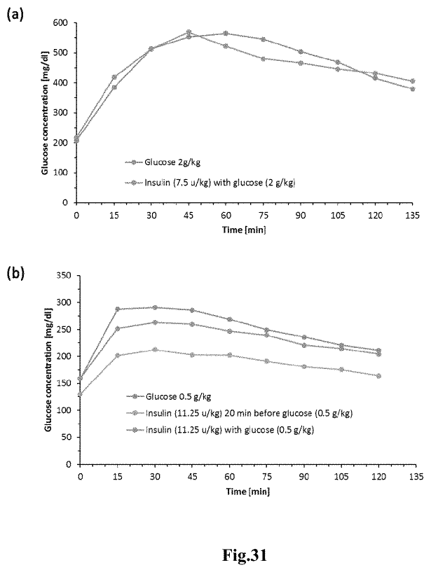

The results of glucose level measurements are shown in Tables 6-10 and in FIG. 12 in a form of graphs presenting mean values in Groups 1 and 2 (FIG. 31a) as well as 3, 4 and 5 (FIG. 31b) with reference to the relevant control group.

TABLE 6
List of results of glucose level measurements in Group 1,
expressed in mg/dl - glucose 2 g/kg only.

| | | Time [min] | | | | | | | | | |
|---|---|---|---|---|---|---|---|---|---|---|---|
| | Mass | Glucose concentration [mg/dl] | | | | | | | | | |
| Lp. | [g] | 0 | 15 | 30 | 45 | 60 | 75 | 90 | 105 | 120 | 135 |
| 1 | 160 | 361 | 481 | 600 | 600 | 600 | 544 | 550 | 494 | 481 | 458 |
| 2 | 163 | 242 | 522 | 600 | 600 | 600 | 600 | 548 | 515 | 431 | 423 |
| 3 | 152 | 188 | 355 | 493 | 516 | 564 | 558 | 500 | 521 | 481 | 445 |
| 4 | 174 | 165 | 350 | 520 | 600 | 600 | 600 | 578 | 516 | 426 | 406 |
| 5 | 178 | 153 | 331 | 436 | 524 | 537 | 512 | 492 | 460 | 416 | 358 |
| 6 | 178 | 138 | 267 | 424 | 476 | 485 | 457 | 357 | 306 | 258 | 185 |

Lp. = No.
Czas [min] = Time [min]
Waga [g] = Weight [g]
Stężenie glukozy [mg/dl] = Glucose concentration [mg/dl]

TABLE 7
List of results of glucose level measurements in Group 2 -
insulin (7.5 u/kg) and glucose (2 g/kg) concurrently.

| | | Time [min] | | | | | | | | | |
|---|---|---|---|---|---|---|---|---|---|---|---|
| | Mass | Glucose concentration [mg/dl] | | | | | | | | | |
| Lp. | [g] | 0 | 15 | 30 | 45 | 60 | 75 | 90 | 105 | 120 | 135 |
| 1 | 167 | 417 | 600 | 600 | 600 | 562 | 517 | 521 | 464 | 436 | 419 |
| 2 | 146 | 238 | 426 | 530 | 600 | 564 | 536 | 494 | 495 | 454 | 460 |
| 3 | 161 | 208 | 470 | 547 | 563 | 530 | 496 | 473 | 495 | 465 | 417 |
| 4 | 164 | 155 | 337 | 455 | 519 | 513 | 461 | 451 | 441 | 442 | 428 |
| 5 | 167 | 141 | 419 | 527 | 527 | 497 | 472 | 480 | 427 | 434 | 384 |
| 6 | 163 | 145 | 259 | 421 | 600 | 465 | 396 | 376 | 353 | 357 | 324 |

TABLE 8
List of results of glucose level measurements in
Group 3 - glucose 0.5 g/kg only.

| | | Time [min] | | | | | | | | |
|---|---|---|---|---|---|---|---|---|---|---|
| | Mass | Glucose concentration [mg/dl] | | | | | | | | |
| Lp. | [g] | 0 | 15 | 30 | 45 | 60 | 75 | 90 | 105 | 120 |
| 1 | 175 | 382 | 569 | 495 | 493 | 495 | 457 | 456 | 415 | 434 |
| 2 | 190 | 155 | 270 | 265 | 289 | 260 | 255 | 222 | 212 | 203 |
| 3 | 166 | 141 | 311 | 317 | 295 | 283 | 274 | 269 | 283 | 263 |
| 4 | 178 | 98 | 208 | 215 | 208 | 186 | 187 | 177 | 161 | 152 |
| 5 | 175 | 98 | 219 | 262 | 255 | 223 | 182 | 170 | 145 | 122 |
| 6 | 184 | 80 | 148 | 190 | 174 | 167 | 141 | 121 | 109 | 93 |

TABLE 9
List of results of glucose level measurements in Group 4 -
insulin (11.25 u/kg) 20 minutes before glucose (0.5 g/kg)

| | | Time [min] | | | | | | | | |
|---|---|---|---|---|---|---|---|---|---|---|
| | Mass | Glucose concentration [mg/dl] | | | | | | | | |
| Lp. | [g] | 0 | 15 | 30 | 45 | 60 | 75 | 90 | 105 | 120 |
| 1 | 180 | 104 | 148 | 145 | 131 | 129 | 112 | 102 | 100 | 92 |
| 2 | 185 | 100 | 187 | 197 | 181 | 193 | 191 | 196 | 173 | 157 |
| 3 | 185 | 120 | 219 | 250 | 254 | 258 | 250 | 234 | 237 | 229 |
| 4 | 182 | 275 | 333 | 337 | 336 | 351 | 350 | 332 | 335 | 304 |
| 5 | 179 | 91 | 163 | 209 | 191 | 173 | 150 | 137 | 129 | 117 |
| 6 | 179 | 90 | 158 | 137 | 122 | 109 | 95 | 86 | 79 | 85 |

TABLE 10
List of results of glucose level measurements in Group 5 -
insulin (11.25 u/kg) and glucose (0.5 g/kg) concurrently

| | | Time [min] | | | | | | | | |
|---|---|---|---|---|---|---|---|---|---|---|
| | Mass | Glucose concentration [mg/dl] | | | | | | | | |
| Lp. | [g] | 0 | 15 | 30 | 45 | 60 | 75 | 90 | 105 | 120 |
| 1 | 180 | 341 | 472 | 452 | 424 | 402 | 403 | 380 | 369 | 333 |
| 2 | 180 | 226 | 301 | 357 | 345 | 347 | 367 | 332 | 337 | 330 |
| 3 | 167 | 110 | 209 | 189 | 189 | 169 | 156 | 144 | 122 | 122 |
| 4 | 166 | 100 | 209 | 216 | 238 | 215 | 194 | 186 | 176 | 179 |
| 5 | 175 | 97 | 171 | 190 | 194 | 194 | 175 | 164 | 158 | 147 |
| 6 | 190 | 83 | 147 | 174 | 166 | 153 | 140 | 119 | 125 | 115 |

Based on the measurements, the surface area below the glucose curve was calculated. Mean value was computed for each group and compared to the relevant control group, whereby the percent proportion was calculated in relation to the control group, i.e. Group 2 to Control Group 1, and Groups 4 and 5 to Control Group 3 (Table 11).

TABLE 11
Results of the measurements of surface areas below the glucose
curve for Groups 2, 4 and 5 (fields P2, P4, P5) by reference to the
relevant control group (P1 and P3).
Percent change in the surface
below the glucose curve (%)

| Group 2 | Group 4 | Group 5 |
|---|---|---|
| (P2/P1)^{a} | (P4/P3)^{a} | (P5/P3)^{a} |
| 84.8 | 61.0 | 76.2 |

^{a}relates to surface areas below glucose curves in Groups 1-5.

### Final Conclusions:

1. The findings show positive effect produced by encapsulated insulin in the glucose curve in animals with streptozotocin-induced type 1 diabetes.
2. The observed effect was more visible in the case of lower glucose dose which suggests a necessity to increase the number of units of insulin in the formulation.
3. More beneficial effect is produced by administration of encapsulated insulin 20 minutes before glucose administration.

1-12. (canceled)

13. A multicompartment system of nanocapsule-in-nanocapsule type, in a form of water-in-oil-in-water double emulsion, for concurrent delivery of hydrophilic and lipophilic compounds, the multicompartment system comprising:
a) a liquid oil core for transport of a lipophilic compound, containing oil selected from the group including: oleic acid, isopropyl palmitate, fatty acids, natural extracts and oils, such as corn oil, linseed oil, soybean oil, argan oil, or their mixtures; beneficially oleic acid;
b) a capsule or many capsules with aqueous core, embedded in an oil core, for transport of a hydrophilic compound;
c) a stabilizing shell for both the capsule with oil core and the inner capsule with water core, consisting of a hydrophobically modified polysaccharide selected from a group comprising: derivatives of chitosan, oligochitosan, dextran, carrageenan, amylose, starch, hydroxypropyl cellulose, pullulan and glycosaminoglycans, hyaluronic acid, heparin sulfate, keratan sulfate, heparan sulfate, chondroitin sulfate, dermatan sulfate; beneficially derivatives of hyaluronic acid;
d) outer capsule with a diameter below 1 μm, stable in aqueous solution; and
e) active substance.

14. The multicompartment system of claim 13, wherein a degree of hydrophobic side chains substitution in a hydrophobically modified polysaccharide ranges from 0.1 to 40%.

15. The multicompartment system of claim 13, wherein the stabilizing shells for the capsule with oil core and the capsule with water core (inner capsule) consist of hydrophobically modified sodium hyaluronate, Hy-Cx, with a formula:

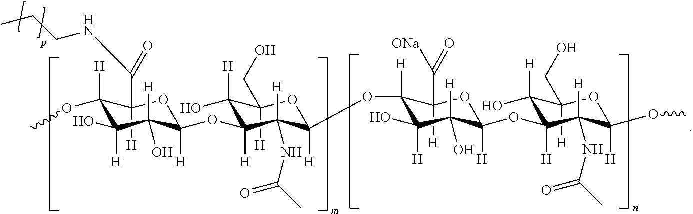

16. The multicompartment system of claim 13, wherein the transported lipophilic compound may be a fluorescent dye, fat-soluble vitamin, or hydrophobic drug.

17. The multicompartment system of claim 13, wherein the transported hydrophilic compound may be a fluorescent dye, water-soluble vitamin, protein or hydrophilic drug; advantageously: insulin.

18. The multicompartment system of claim 17, wherein insulin is in a concentration of 0.005-20.000 of insulin units per 1 ml of the capsule suspension.

19. A method of producing a multicompartment system of nanocapsule-in-nanocapsule type, in the form of water-in-oil-in-water double emulsion, as defined in claim 13, the method comprising:
a) during the first step invert emulsion of water-in-oil (W/O) type is produced by mixing an aqueous solution of hyaluronic acid dodecyl derivative Hy-Cx, described by the above formula, with a non-toxic oil constituting about 0.1-99.9% of the mixture volume, by exposition to ultrasounds (sonication) or to mechanical stimuli, advantageously—mixing or shaking, with aqueous phase to oil phase volume ratio ranging from 1:10 to 1:10000; advantageously approx. 1:100;
b) during the second step, water droplets suspended in the continuous oil phase receive hyaluronate coating, with W/O phase emulsion to aqueous phase volume ratio ranging from 1:10 to 1:10000; advantageously approx. 1:100; and
c) as a result, the water-in-oil-in-water (W/O/W) double emulsion system is produced by exposition to ultrasounds (sonication) or to mechanical stimuli, advantageously—mixing or shaking,
wherein, the aqueous phase applied is based on aqueous solution of hydrophobically modified polysaccharide selected from a group comprising: derivatives of chitosan, oligochitosan, dextran, carrageenan, amylose, starch, hydroxypropyl cellulose, pullulan and glycosaminoglycans, and particularly hyaluronic acid, heparin sulfate, keratan sulfate, heparan sulfate, chondroitin sulfate, dermatan sulfate; advantageously derivatives of hyaluronic acid with pH in the range of 2-12, concentration of 0.1-30 g/L and ionic strength in the range of 0.001-3 mol/dm³,
and the oil phase contains oil selected from the group including: oleic acid, isopropyl palmitate, fatty acids, natural oils, in particular linseed oil, soybean oil, argan oil, or their mixtures; beneficially oleic acid,
notably, the process is carried out without using any small-particle surfactants.

20. The method of claim 19, wherein pulsed sonication is carried out with impulse duration twice as short as the duration of the interval between two consecutive impulses.

21. The method of claim 19, wherein the encapsulated lipophilic compound is contained in the oil core and the encapsulated hydrophilic compound is comprised in the water core of the nanocapsule.

22. The method of claim 19, wherein the content of ionic groups in the polysaccharide is not lower than 20 mol %, and is greater than 60 mol-% (calculated per one mer).

23. The method of claim 19, wherein during the first and second step, sonication is continued for 15-60 minutes, at a temperature of 18° C.-40° C., for at least 60 min to obtain invert emulsion, and at least 30 min to obtain double emulsion, at a temperature of 25-30° C.

24. Application of the multicompartment system of claim 13, for transport of lipophilic compounds and hydrophilic compounds, where the lipophilic compound may be a fluorescent dye, fat-soluble vitamin, or a hydrophobic drug, while the hydrophilic compound may be a fluorescent dye, water-soluble vitamin, protein or a hydrophilic drug.
